(19) United States
(12) Patent Application Publication
(10) Pub. No.: US 2006/0241166 A1
(43) Pub. Date: Oct. 26, 2006

(54) Sapphyrins and uses thereof

(75) Inventor: Darren Magda, Cupertino, CA (US)

Correspondence Address: WILSON SONSINI GOODRICH & ROSATI, 650 PAGE MILL ROAD, PALO ALTO, CA, 94304-1050, US

(73) Assignee: Pharmacyclica, Inc.

(21) Appl. No.: 10/552,696

(22) PCT Filed: Apr. 5, 2004
(86) PCT No.: PCT/US04/10481

§ 371 Date: Jul. 13, 2006

Related U.S. Application Data
(60) Provisional application No. 60/460,846, filed on Apr. 4, 2003.
(60) Provisional application No. 60/520,275, filed on Nov. 13, 2003.
(60) Provisional application No. 60/527,510, filed on Dec. 5, 2003.

(51) Int. Cl. A61K 31/409; C07D 487/22

(52) U.S. Cl. 514/410; 540/472

(57) ABSTRACT

The present invention relates to compounds of Formula (I): their pharmaceutical composition and their utility in treating neoplasm.

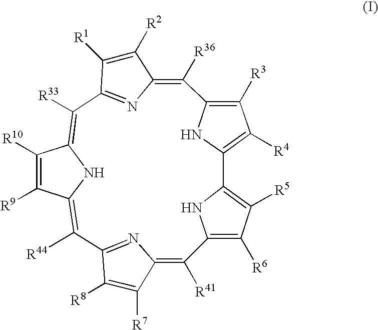

### CLAIM OF PRIORITY

This PCT International patent application claims the benefit of priority from U.S. Provisional Patent Application Ser. No. 60/460,846, filed Apr. 4, 2003 (Attorney Docket No. 4239.00 US), U.S. Provisional Patent Application Ser. No. 60/520,275, filed Nov. 13, 2003 (Attorney Docket No. 4241.00 US), and U.S. Provisional Patent Application Ser. No. 60/527,510, filed Dec. 5, 2003 (Attorney Docket No. 4242.00 US), all of which are incorporated herein by reference in their entirety.

### FIELD OF INVENTION

The present invention relates to sapphyrin compounds of Formula I and their utility as anticancer agents.

### BACKGROUND OF INVENTION

Sapphyrins, are molecules that have been extensively studied by Sessler et al., Sessler, J. L.; Davis, J. M. “Sapphyrins: Versatile Anion-binding Agents,” Acc. Chem. Res., vol. 34, pgs. 989-997 (2001). In early work, Shionoya, M.; Furuta, H.; Lynch, V.; Harriman, A.; Sessler, J. L. “Diprotonated Sapphyrin: A Fluoride Selective Halide Anion Receptor,” J. Am. Chem. Soc., vol. 114, pgs. 5714-5722 (1992), Prof. Sessler and his coworkers established that, in marked contrast to porphyrins, sapphyrins are readily protonated and form well-defined anion complexes in the solid state. None of the sapphyrin work suggests any utility of sapphyrins to treat neoplasm.

### SUMMARY OF THE INVENTION

The present invention provides compounds of Formula I: 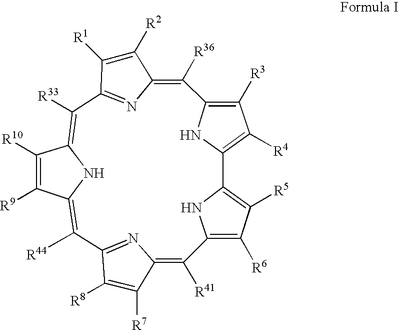
its pharmaceutically acceptable salts and prodrugs thereof, wherein:
R¹ represents —(CH₂)₁₋₄—X—CH₂—O—(CH₂CH₂O)₀₋₃—CH₃, —C₁₋₄ alkyl, —(CH₂)₁₋₄—R²¹, H or —R²¹, —(CH₂)₁₋₄—O—C(═O)—NR³¹R³², or —(CH₂)₁₋₄—OH;
R² represents H, —C₁₋₄ straight chain alkyl, or —C₃₋₆ branched alkyl;
R³ represents H, —C₁₋₄ straight chain alkyl, —C₃₋₆ branched alkyl, halogen, —NO₂, —CN, —O-alkyl, —(CH₂)₁₋₄O—(CH₂)₁₋₄O—(CH₂)₁₋₄O—(CH₂)₀₋₂—CH₃, —(CH₂)₁₋₄—OH, or —(CH₂)₁₋₄—OCOCH₃;
R⁴ represents H, —C₁₋₄ straight chain alkyl, —C₃₋₆ branched alkyl, halogen, —CN, —O-alkyl, —(CH₂)₁₋₄—OH, —(CH₂)₁₋₄O—(CH₂)₁₋₄O—(CH₂)₁₋₄O—(CH₂)₀₋₂—CH₃, —NO₂, or —(CH₂)₁₋₄—OCOCH₃;
R⁵ represents H, —C₁₋₄ straight chain alkyl, —C₃₋₆ branched alkyl, halogen, —CN, —O-alkyl, —(CH₂)₁₋₄—OH, —(CH₂)₁₋₄O—(CH₂)₁₋₄O—(CH₂)₁₋₄O—(CH₂)₀₋₂—CH₃, —NO₂, or —(CH₂)₁₋₄—OCOCH₃;
R⁶ represents H, —C₁₋₄ straight chain alkyl, —C₃₋₆ branched alkyl, halogen, —CN, O-alkyl, —(CH₂)₁₋₄—OH, —(CH₂)₁₋₄O—(CH₂)₁₋₄O—(CH₂)₁₋₄O—(CH₂)₀₋₂—CH₃, —NO₂, or —(CH₂)₁₋₄—OCOCH₃;
R⁷ represents H, —C₁₋₄ straight chain alkyl, or —C₃₋₆ branched alkyl;
R⁸ represents —(CH₂)₁₋₄—X—CH₂—O—(CH₂CH₂O)₀₋₃—CH₃, —C₁₋₄ alkyl, —(CH₂)₁₋₄—R²¹, —R²¹, H, —(CH₂)₁₋₄—O—C(═O)—NR³¹R³² or —(CH₂)₁₋₄—OH;
R⁹ represents —C₁₋₄ straight chain alkyl, —C₃₋₆ branched alkyl, H, —O—C₁₋₄-alkyl, —O—C₃₋₆ branched alkyl, or —(CH₂)₁₋₄O—(CH₂)₁₋₄O—(CH₂)₁₋₄O—(CH₂)₀₋₂—CH₃;
R¹⁰ represents H, —C₁₋₄ straight chain alkyl, —C₃₋₆ branched alkyl, —O—C₁₋₄-alkyl, or —O—C₃₋₆ branched alkyl;
X represents —OCO₂CH₂—, —O₂C—, —NHCO—, —OCONHCH₂, —NHCO₂CH₂—, —NHCONHCH₂—, or —NHCH₂—;
R²¹ R²², R²³, R²⁴, and R²⁵ independently at each occurrence are selected from H, —CH₂OH, —CH₂NH₂, —CH₂N(C₂H₄OH)₂, —COOH, —CON(C₂H₄OH)₂, —OCON(C₂H₄OH)₂, —NHCON(C₂H₄OH)₂, and —O(CH₂CH₂O)₀₋₃CH₃;
R³¹ represents H, —(CH₂)₁₋₆OH, C((CH₂)₁₋₄OH)₃, —C((CH₂)₁₋₄O-alkyl)₃, —(CH₂)₁₋₆O-alkyl, or —(CH₂)₁₋₄O—(CH₂)₁₋₄O—(CH₂)₁₋₄O—(CH₂)₀₋₂—CH₃;
R³² represents H, —(CH₂)₁₋₆OH, C((CH₂)₁₋₄OH)₃, —C((CH₂)₁₋₄O-alkyl)₃, —(CH₂)₁₋₆O-alkyl, or —(CH₂)₁₋₄O—(CH₂)₁₋₄O—(CH₂)₁₋₄O—(CH₂)₀₋₂—CH₃;
R³³ represents H, —C₁₋₄ alkyl, —O—C₁₋₄-alkyl, —O—C₃₋₆ branched alkyl, or 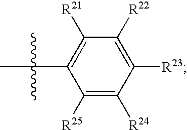
R³⁶ represents H or —C₁₋₄ alkyl;
R³⁷ represents H or —C₁₋₄ alkyl;
R⁴¹ represents H or —C₁₋₄ alkyl; and
R⁴⁴ represents H, —C₁₋₄ alkyl, —O—C₁₋₄ alkyl, or

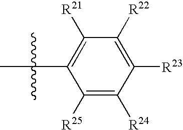

The present invention also provides a method of treating a host harboring a neoplasm or atheroma comprising administering to the host a compound of Formula I.

### DETAILED DESCRIPTION

The present invention provides a compound of Formula I: 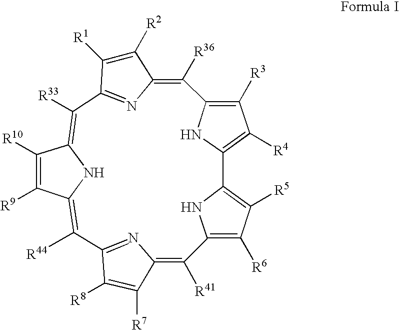
its pharmaceutically acceptable salts and prodrugs there of, wherein:
R¹ represents —(CH₂)₁₋₄—O—C(═O)—NR³¹R³²—(CH₂)₁₋₄—X—CH₂—O—(CH₂CH₂O)₀₋₃—CH₃, —C₁₋₄ alkyl, —(CH₂)₁₋₄—R²¹, H or —R²¹ or —(CH₂)₁₋₄—OH;
R² represents H, —C₁₋₄ straight chain alkyl, or —C₃₋₆ branched alkyl;
R³ represents H, —C₁₋₄ straight chain alkyl, —C₃₋₆ branched alkyl, halogen, —NO₂, CN, O-alkyl, —(CH₂)₁₋₄O—(CH₂)₁₋₄O—(CH₂)₁₋₄O—(CH₂)₀₋₂—CH₃, —(CH₂)₁₋₄—OH, or (CH₂)₁₋₄—OCOCH₃;
R⁴ represents H, C₁₋₄ straight chain alkyl, C₃₋₆ branched alkyl, halogen, —NO₂, —CN, —O-alkyl, —(CH₂)₁₋₄—OH, —(CH₂)₁₋₄O—(CH₂)₁₋₄O—(CH₂)₁₋₄O—(CH₂)₀₋₂—CH₃, or —(CH₂)₁₋₄—OCOCH₃;
R⁵ represents H, —C₁₋₄ straight chain alkyl, —C₃₋₆ branched alkyl, halogen, —NO₂, —CN, —O-alkyl, —(CH₂)₁₋₄—OH, —(CH₂)₁₋₄O—(CH₂)₁₋₄O—(CH₂)₁₋₄O—(CH₂)₀₋₂—CH₃, or —(CH₂)₁₋₄—OCOCH₃;
R⁶ represents H, —C₁₋₄ straight chain alkyl, —C₃₋₆ branched alkyl, halogen, —NO₂, —CN, —O-alkyl, —(CH₂)₁₋₄—OH, (CH₂)₁₋₄O—(CH₂)₁₋₄O—(CH₂)₁₋₄O—(CH₂)₀₋₂—CH₃, or —(CH₂)₁₋₄—OCOCH₃;
R⁷ represents H, —C₁₋₄ straight chain alkyl, or —C₃₋₆ branched alkyl;
R⁸ represents —(CH₂)₁₋₄—X—CH₂—O—(CH₂CH₂O)₀₋₃—CH₃, —C₁₋₄ alkyl, —(CH₂)₁₋₄—R²¹, —R²¹, H, —(CH₂)₁₋₄—O—C(═O)—NR³¹R³² or —(CH₂)₁₋₄—OH;
R⁹ represents —C₁₋₄ straight chain alkyl, —C₃₋₆ branched alkyl, H, —O—C₁₋₄-alkyl, —O—C₃₋₆ branched alkyl, or —(CH₂)₁₋₄O—(CH₂)₁₋₄O—(CH₂)₁₋₄O—(CH₂)₀₋₂—CH₃;
R¹⁰ represents H, —C₁₋₄ straight chain alkyl, —C₃₋₆ branched alkyl, —O—C₁₋₄-alkyl, or —O—C₃₋₆ branched alkyl;
X represents —OCO₂CH₂—, —O₂C—, —NHCO—, —OCONHCH₂, —NHCO₂CH₂—, —NHCONHCH₂—, or —NHCH₂—;
R²¹ R²², R²³, R²⁴, and R²⁵ independently at each occurrence are selected from H, —CH₂OH, —CH₂NH₂, —CH₂N(C₂H₄OH)₂, —COOH, —CON(C₂H₄OH)₂, —OCON(C₂H₄OH)₂, —NHCON(C₂H₄OH)₂, and —O(CH₂CH₂O)₀₋₃CH₃;
R³¹ represents H, —(CH₂)₁₋₆OH, C((CH₂)₁₋₄OH)₃, —C((CH₂)₁₋₄O-alkyl)₃, —(CH₂)₁₋₆O-alkyl, or (CH₂)₁₋₄O—(CH₂)₁₋₄O—(CH₂)₁₋₄O—(CH₂)₀₋₂—CH₃;
R³² represents H, —(CH₂)₁₋₆OH, C((CH₂)₁₋₄OH)₃, —C((CH₂)₁₋₄O-alkyl)₃, —(CH₂)₁₋₆O-alkyl, or —(CH₂)₁₋₄O—(CH₂)₁₋₄O—(CH₂)₁₋₄O—(CH₂)₀₋₂—CH₃;
R³³ represents H, —C₁₋₄ alkyl, —O—C₁₋₄-allyl, —O—C₃₋₆ branched alkyl, or 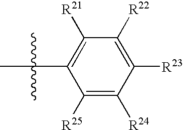
R³⁶ represents H or —C₁₋₄ alkyl;
R³⁷ represents H or —C₁₋₄ alkyl;
R⁴¹ represents H or —C₁₋₄ alkyl; and
R⁴⁴ represents H, —C₁₋₄ alkyl, —O—C₁₋₄ alkyl, or

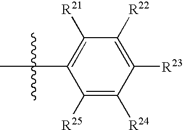

A preferred embodiment provides a compound of Formula I wherein:

R¹ represents —(CH₂)₃—O—C(═O)—NR³¹R³²;

R² represents C₁₋₄ straight chain alkyl, or —C₃₋₆ branched alkyl;

R³ represents —C₁₋₄ straight chain alkyl, —(CH₂)₁₋₄O—(CH₂)₁₋₄O—(CH₂)₁₋₄O—(CH₂)₀₋₂—CH₃, —C₃₋₆ branched alkyl, halogen, —O-alkyl, —(CH₂)₁₋₄—OH, or (CH₂)₁₋₄—OCOCH₃;

R⁴ represents C₁₋₄ straight chain alkyl, —C₃₋₅ branched alkyl, halogen, —(CH₂)₁₋₄—OH, or (CH₂)₁₋₃—OCOCH₃;

R⁵ represents —C₁₋₃ straight chain alkyl, —C₃₋₅ branched alkyl, halogen, —O-alkyl, —(CH₂)₁₋₃—OH, —(CH₂)₁₋₄—(CH₂)₁₋₄O—(CH₂)₁₋₄O—(CH₂)₀₋₂—CH₃, or —(CH₂)₁₋₃—OCOCH₃;

R⁶ represents C₁₋₃ straight chain alkyl, —C₃₋₅ branched alkyl, halogen, —O-alkyl, —(CH₂)₁₋₃—OH, —(CH₂)₁₋₃O—(CH₂)₁₋₄O—(CH₂)₁₋₄O—(CH₂)₀₋₂—CH₃, or —(CH₂)₁₋₄—OCOCH₃;

R⁷ represents —C₁₋₃ straight chain alkyl, or —C₃₋₅ branched alkyl;

R⁸ represents —(CH₂)₂₋₄—O—C(═O)—NR³¹R³²;

R⁹ represents —C₁₋₃ straight chain alkyl, C₃₋₅ branched alkyl, —(CH₂)₂₋₄O—(CH₂)₁₋₄O—(CH₂)₁₋₄O—(CH₂)₀₋₂—CH₃, or —O-alkyl;

R¹⁰ represents —C₁₋₄ straight chain alkyl, C₃₋₆ branched alkyl, or —O-alkyl;

R³¹ represents H, or —(CH₂)₂₋₄O—(CH₂)₁₋₄O—(CH₂)₁₋₄O—(CH₂)₀₋₂—CH₃; and

R³² represents H, or —(CH₂)₂₋₄O—(CH₂)₁₋₄O—(CH₂)₁₋₄O—(CH₂)₀₋₂—CH₃.

Another preferred embodiment provides a compound of Formula I wherein:

R² represents —CH₃;

R³ represents —CH₃, —C₂H₅, or —OCH₃;

R⁴ represents —CH₃, or —C₂H₅;

R⁵ represents —CH₃, —C₂H₅, or —OCH₃;

R⁶ represents —CH₃, —C₂H₅, or —OCH₃;

R⁷ represents —CH₃;

R⁹ represents —CH₃, —C₂H₅, or —OCH₃;

R¹⁰ represents —CH₃, —C₂H₅, or —OCH₃;

R³¹ represents —(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H.

A further preferred embodiment provides a compound of Formula I wherein:

R¹ represents —(CH₂)₃—O—C(═O)—NR³¹R³²;

R² represents —CH₃;

R³ represents —CH₃, or —C₂H₅;

R⁴ represents —CH₃, or —C₂H₅;

R⁵ represents —CH₃, or —C₂H₅;

R⁶ represents —CH₃, —C₂H₅, or —OCH₃;

R⁷ represents —CH₃;

R⁹ represents —CH₃, —C₂H₅, or —OCH₃;

R¹⁰ represents —CH₃, —C₂H₅, or —OCH₃;

R³¹ represents-(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H.

Particularly preferred embodiments provide compounds of Formula I wherein:

R¹ represents —(CH₂)₁₋₃—O—C(═O)—NR³¹R³²;

R² represents —CH₃;

R³ represents —C₂H₅;

R⁴ represents —CH₃;

R⁵ represents —CH₃;

R⁶ represents —C₂H₅;

R⁷ represents —CH₃;

R⁸ represents —(CH₂)₁₋₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

Another particularly preferred embodiment provides a compound of Formula I wherein:

R¹ represents —(CH₂)₁₋₃—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₁₋₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

Yet another preferred embodiment provides a compound of Formula I wherein:

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents —(CH₂)₂OH;

R³² represents —(CH₂)₂OH; and

R³³, R³⁶, R⁴¹ and R⁴⁴ represent H.

Another aspect of the present invention provides a pharmaceutical composition, comprising a pharmaceutically acceptable carrier and a therapeutically effective amount of a compound of Formula I, or a pharmaceutically acceptable salt form thereof. Another embodiment of this aspect of the invention provides a pharmaceutical composition, comprising a pharmaceutically acceptable carrier and a therapeutically effective amount of a compound of Formula I, wherein:

R¹ represents —(CH₂)₂—O—C(═O)—NR³¹R³²;

R² represents —CH₃;

R³ represents —CH₃, —C₂H₅, or —OCH₃;

R⁴ represents —CH₃, or —C₂H₅;

R⁵ represents —CH₃, —C₂H₅, or —OCH₃;

R⁶ represents —CH₃, —C₂H₅, or —OCH₃;

R⁷ represents —CH₃;

R⁸ represents —(CH₂)₂—O—C(═O)—NR³¹R³²;

R⁹ represents —CH₃, —C₂H₅, or —OCH₃;

R¹⁰ represents —CH₃, —C₂H₅, or —OCH₃;

R³¹ represents —(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H; or a pharmaceutically acceptable salt form thereof.

Yet another embodiment of this aspect of the invention provides a pharmaceutical composition, comprising a pharmaceutically acceptable carrier and a therapeutically effective amount of a compound of Formula I, wherein:

R¹ represents —(CH₂)₂—O—C(═O)—NR³¹R³²;

R² represents —CH₃;

R³ represents —C₂H₅, or —OCH₃;

R⁴ represents —CH₃;

R⁵ represents —CH₃;

R⁶ represents —C₂H₅, or —OCH₃;

R⁷ represents —CH₃;

R⁸ represents —(CH₂)₂—O—C(═O)—NR³¹R³²;

R⁹ represents —CH₃, —C₂H₅, or —OCH₃;

R¹⁰ represents —CH₃, —C₂H₅, or —OCH₃;

R³¹ represents —(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H.

Yet another embodiment of this aspect of the invention provides a pharmaceutical composition, comprising a pharmaceutically acceptable carrier and a therapeutically effective amount of a compound of Formula I, wherein:

R¹ represents —(CH₂)₂—O—C(═O)—NR³¹R³²;

R² represents —CH₃;

R³ represents —C₂H₅;

R⁴ represents —CH₃;

R⁵ represents —CH₃;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents —(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H.

Yet another embodiment of this aspect of the invention provides a pharmaceutical composition, comprising a pharmaceutically acceptable carrier and a therapeutically effective amount of a compound of Formula I, wherein:

R¹ represents —(CH₂)₁₋₃—O—C(═O)—NR³¹R³²;

R² represents —CH₃;

R³ represents —C₂H₅;

R⁴ represents —CH₃;

R⁵ represents —CH₃;

R⁶ represents —C₂H₅;

R⁷ represents —CH₃;

R⁸ represents —(CH₂)₁₋₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

Yet another embodiment of this aspect of the invention provides a pharmaceutical composition, comprising a pharmaceutically acceptable carrier and a therapeutically effective amount of a compound of Formula I, wherein:

R¹ represents —(CH₂)₁₋₃—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₁₋₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

Another aspect of the present invention provides a method of treating a host harboring a neoplasm comprising administering to the host a therapeutically effective amount of a compound of Formula I, or a pharmaceutically acceptable salt form thereof. Another embodiment of this aspect of the invention provides a method of treating a host harboring a neoplasm comprising administering to the host a therapeutically effective amount of a compound of Formula I, or a pharmaceutically acceptable salt form thereof, wherein:

R¹ represents —(CH₂)₂—O—C(═O)—NR³¹R³²;

R² represents —CH₃;

R³ represents —CH₃, —C₂H₅, or —OCH₃;

R⁴ represents —CH₃, or —C₂H₅;

R⁵ represents —CH₃, —C₂H₅, or —OCH₃;

R⁶ represents —CH₃, —C₂H₅, or —OCH₃;

R⁷ represents —CH₃;

R⁸ represents —(CH₂)₂—O—C(═O)—NR³¹R³²;

R⁹ represents —CH₃, —C₂H₅, or —OCH₃;

R¹⁰ represents —CH₃, —C₂H₅, or —OCH₃;

R³¹ represents —(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H; or a pharmaceutically acceptable salt form thereof.

Yet another embodiment of this aspect of the invention provides a method of treating a host harboring a neoplasm comprising administering to the host a therapeutically effective amount of a compound of Formula I, or a pharmaceutically acceptable salt form thereof, wherein:

R¹ represents —(CH₂)₂—O—C(═O)—NR³¹R³²;

R² represents —CH₃;

R³ represents —C₂H₅, or —OCH₃;

R⁴ represents —CH₃;

R⁵ represents —CH₃;

R⁵ represents —C₂H₅, or —OCH₃;

R⁷ represents —CH₃;

R⁸ represents —(CH₂)₂—O—C(═O)—NR³¹R³²;

R⁹ represents —CH₃, —C₂H₅, or —OCH₃;

R¹⁰ represents —CH₃, —C₂H₅, or —OCH₃;

R³¹ represents —(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H.

Yet another embodiment of this aspect of the invention provides a method of treating a host harboring a neoplasm comprising administering to the host a therapeutically effective amount of a compound of Formula I, or a pharmaceutically acceptable salt form thereof, wherein:

R¹ represents —(CH₂)₂—O—C(═O)—NR³¹R³²;

R² represents —CH₃;

R³ represents —C₂H₅;

R⁴ represents —CH₃;

R⁵ represents —CH₃;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents —(CH₂)₂—O—(CH₂)₂—O—(CH₂)₂—O—CH₃;

R³² represents —(CH₂)₂—O—(CH₂)₂—O—(CH₂)₂—O—CH₃; and

R³³, R³⁶, R⁴¹ and R⁴⁴ represent H.

Yet another embodiment of this aspect of the invention provides a method of treating a host harboring a neoplasm comprising administering to the host a therapeutically effective amount of a compound of Formula I, or a pharmaceutically acceptable salt form thereof, wherein:

R¹ represents —(CH₂)₁₋₃—O—C(═O)—NR³¹R³²;

R² represents —CH₃;

R³ represents —C₂H₅;

R⁴ represents —CH₃;

R⁵ represents —CH₃;

R⁶ represents —C₂H₅;

R⁷ represents —CH₃;

R⁸ represents —(CH₂)₁₋₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

Yet another embodiment of this aspect of the invention provides a method of treating a host harboring a neoplasm comprising administering to the host a therapeutically effective amount of a compound of Formula I, or a pharmaceutically acceptable salt form thereof, wherein:

R¹ represents —(CH₂)₁₋₃—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₁₋₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

### DETAILED DESCRIPTION OF FIGURES

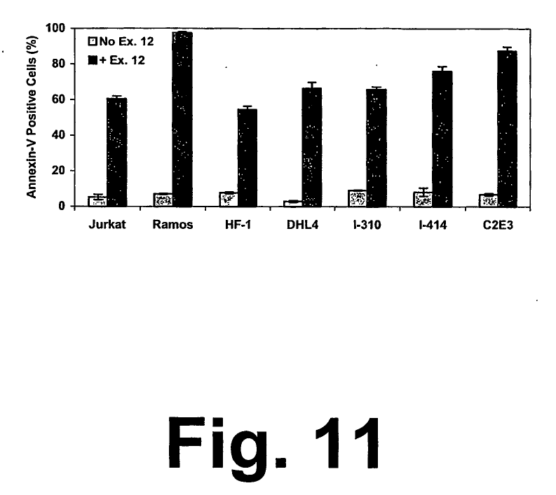

FIG. 11 shows the effect of 1 μM Example 12 on Lymphoma, Leukemia and Myeloma cell lines when tested for cell death. Adding compound of Example 12 causes at least a five-fold increase in cell death in cell lines tested.

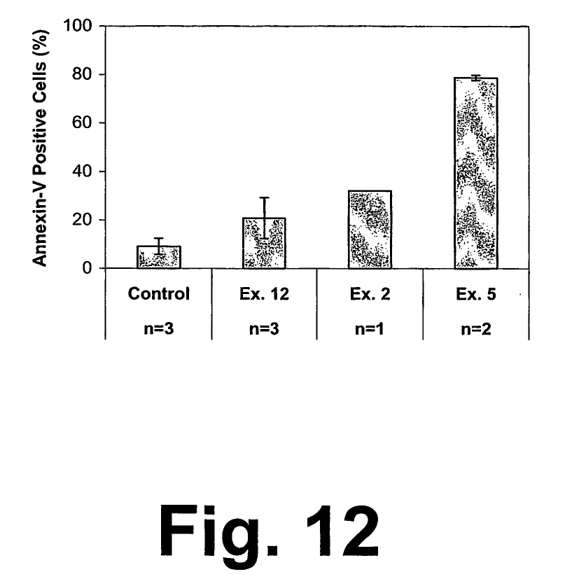

FIG. 12 shows effect of various sapphyrins of Formula I when added to Ramos Xenograft cells. Tumor cells were extracted from animals and tested for cell death. Compound of Example 5 caused the most cell death in this model.

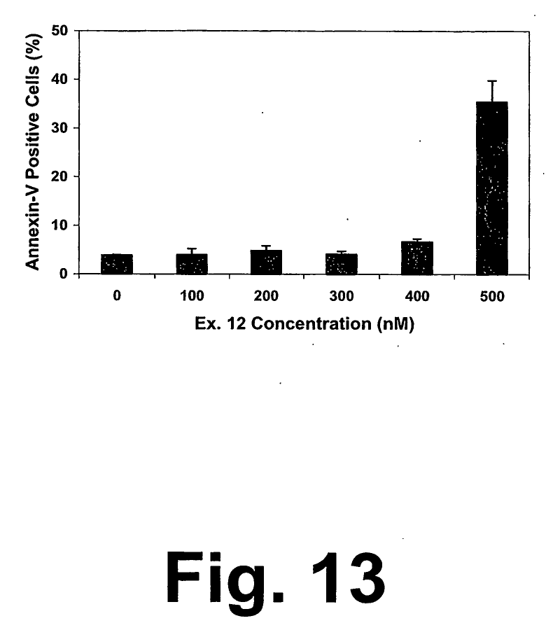

FIG. 13 shows dose response of Example 12 in Ramos cell lines after 48 hours incubation. An increase in the amount of Example 12 causes an increase in cell death.

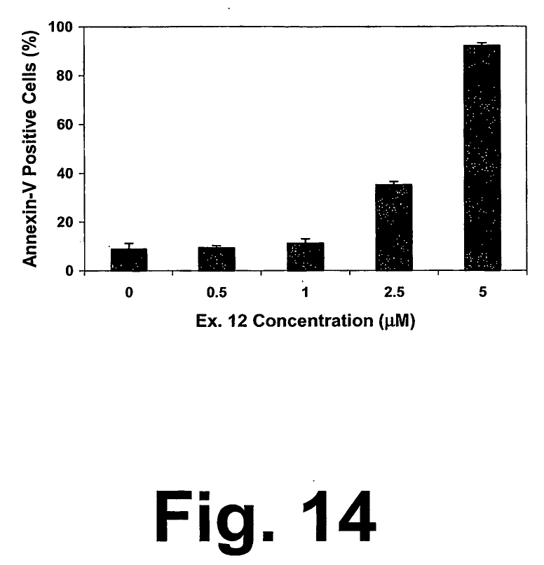

FIG. 14 shows a dose response of Example 12 in Ramos cell line after 8 hours. An increase in the amount of Example 12 causes an increase in cell death.

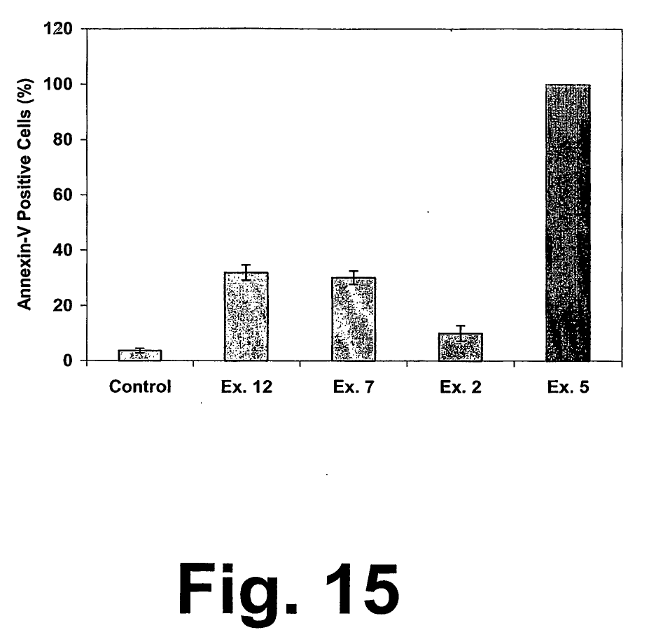

FIG. 15 shows the effect of various sapphyrins when added to Ramos cells in causing cell death. The amount added was 11 μM each and Example 5 shows leads to most cell death after 24 hours.

### EXPERIMENTAL

Preparation of Dihydroxysapphyrin

In a 3L three-neck round bottom flask, were placed TP4 (963 mg, 2.0 mmol), 3,4-diethylpyrrole (493 mg, 4.0 mmol), CH₂Cl₂ (2000 mL), and a magnetic stir bar. With stirring, trifluoroacetice acid (100 mL) was added to the flask, and the reaction mixture was stirred for 48 hr at room temperature. Then triethylamine (180 mL) was added dropwise to the solution. The resulting mixture was concentrated on a rotary evaporator to a volume of about 500 mL and then extracted with water three times (100 mL, 200 mL, and 200 mL) using a separation funnel. The organic phase (methylene chloride solution) was directly loaded to a neutral aluminum oxide column. The column was first eluted with 1% MeOH/CH₂Cl₂ to separate a red-colored band (porphyrin byproduct). After the red band was eluted, the polarity was increased to 5% MeOH/CH₂Cl₂ to elute the green band (sapphyrin product). The sapphyrin fraction was concentrated to give dihydroxysapphyrin as a shiny blue solid (304 mg, 22%). 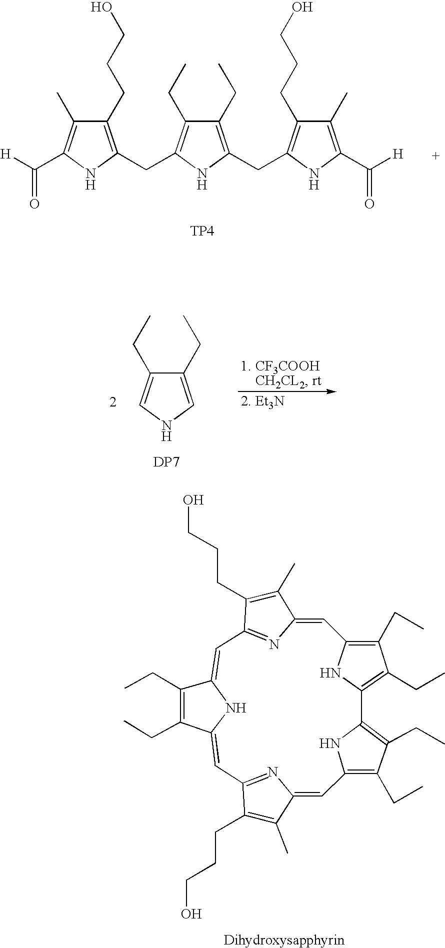
Preparation of Carbamate-linked Tetrahydroxy Sapphyrin

In a 25 mL Schlenk tube were placed bishydroxypropyl sapphyrin (100 mg, 0.145 mmol), N,N′-disuccinimidyl carbonate (186 mg, 0.725 mmol), and a magnetic stir bar. The system was dried in vacuum at rt for 2 hrs. Under a stream of N₂, anhydrous CH₂Cl₂ (5 mL) and diisopropylethylamine (DIEA, 187 mg, 1.45 mmol) were added. The reaction mixture was stirred at rt for 4 hrs. Then diethanolamine (152 mg, 1.45 mmol, dissolved in 1 mL CH₂Cl₂) was added, and the resulting mixture was stirred for another 1 hr. The reaction mixture was concentrated to give an oily residue, which was purified by column chromatography on silica gel column (eluent: 10-15% MeOH in CH₂Cl₂ with 0.5% HOAc) to yield a blue solid. This crude product was then dissolved in a mixture of 1 mL MeOH and 4 mL DI water, loaded on a Sep-Pak. After washing with 30 mL DI water, the product band was eluted with MeOH containing 2% HOAc. Concentration of the MeOH solution gave tetrahydroxy carbamate sapphyrin (mono acetate form, 75 mg, 51%). 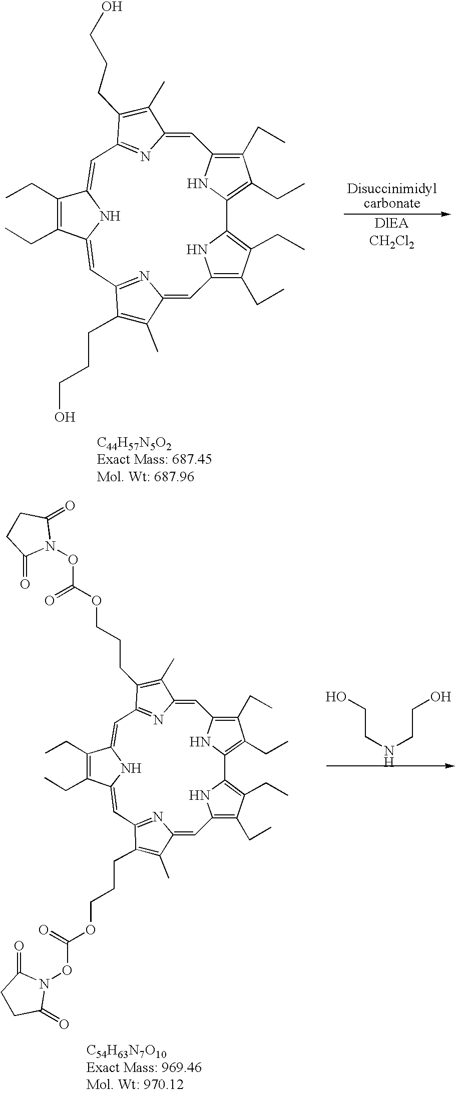 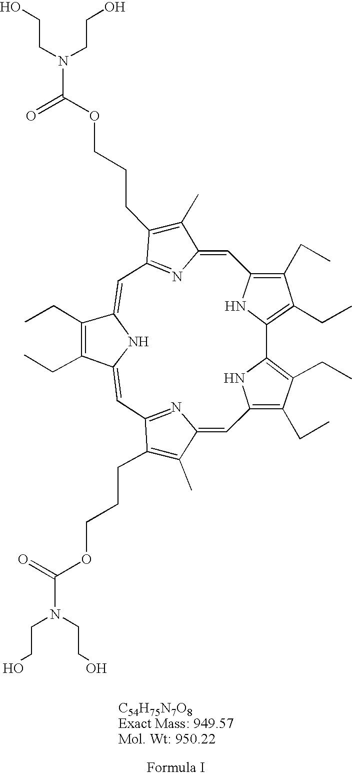
Following Formula I compounds were synthesized using the above procedures:

### Example 1

R¹ represents —(CH₂)₃—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents —(CH₂)₂OH;

R³² represents —(CH₂)₂OH; and

R³³, R³⁶, R⁴¹ and R⁴⁴ represent H.

### Example 2

R¹ represents —(CH₂)₃—O—C(═O)—NR³¹R³²;

R² represents —CH₃;

R³ represents —C₂H₅;

R⁴ represents —C₂H₅;

R⁵ represents —C₂H₅;

R⁵ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents H;

R³² represents —C(CH₂—OH)₃; and

R³³, R³⁶, R⁴¹ and R⁴⁴ represent H.

### Example 3

R¹ represents —(CH₂)₃—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents —(CH₂—CH₂O)₃CH₃;

R³² represents H; and

R³³, R³⁶, R⁴¹ and R⁴⁴ represent H.

### Example 4

R¹ represents —(CH₂)₃—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents —CH₂—(CH₂OCH₂)₄₋₅CH₂—O—CH₃;

R³² represents H; and

R³³, R³⁶, R⁴¹ and R⁴⁴ represent H.

### Example 5

R¹ represents —(CH₂)₃—O—C(═O)—NR³¹R³²;

R² represents —CH₃;

R³ represents —C₂H₅;

R⁴ represents —CH₃;

R⁵ represents —CH₃;

R⁶ represents —C₂H₅;

R⁷ represents —CH₃;

R⁸ represents —(CH₂)₁₋₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

### Example 6

R¹ represents —(CH₂)₃—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents —(CH₂—CH₂O)₃CH₃;

R³² represents —(CH₂—CH₂O)₃CH₃; and

R³³, R³⁶, R⁴¹ and R⁴⁴ represent H.

### Example 7

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents —(CH₂)₂OH;

R³² represents —(CH₂)₂OH; and

R³³, R³⁶, R⁴¹ and R⁴⁴ represent H.

### Example 11

R¹ represents —(CH₂)₃—OH;

R² represents —CH₃;

R³ represents —C₂H₅;

R⁴ represents —CH₃;

R⁵ represents —CH₃;

R⁶ represents —C₂H₅;

R⁷ represents —CH₃;

R⁸ represents —(CH₂)₃—OH;

R⁹ represents —C₂H₅;

R¹⁰ represents —C₂H₅; and

R³³, R³⁶, R⁴¹ and R⁴⁴ represent H.

### Example 12

R¹ represents —(CH₂)₃—OH;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—OH;

R⁹ represents —C₂H₅;

R¹⁰ represents —C₂H₅; and

R³³, R³⁶, R⁴¹ and R⁴⁴ represent H.

### Example 22

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents H;

R³² represents —C(CH₂—OH)₃; and

R³³, R³⁶, R⁴¹ and R⁴⁴ represent H.

### Example 23

R¹ represents —(CH₂)₁—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₁—O—C(═O)—NR³¹R³²;

R⁹ represents —C₂H₅;

R¹⁰ represents —C₂H₅;

R³¹ represents H;

R³² represents —C(CH₂—OH)₃; and

R³³, R³⁶, R⁴¹ and R⁴⁴ represent H.

### Example 24

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents H;

R³² represents —C(CH₂—OH)₃; and

R³³, R³⁶, R⁴¹ and R⁴⁴ represent H.

### Example 33

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents —(CH₂—CH₂O)₃CH₃;

R³² represents H; and

R³³, R³⁶ R⁴¹ and R⁴⁴ represent H.

### Example 34

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents —(CH₂—CH₂O)₃CH₃;

R³² represents H; and

R³³, R³⁶, R⁴¹ and R⁴⁴ represent H.

### Example 35

R¹ represents —(CH₂)₁—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₁—O—C(═O)—NR³¹R³²;

R⁹ represents —C₂H₅;

R¹⁰ represents —C₂H₅;

R³¹ represents —(CH₂—CH₂O)₃CH₃;

R³² represents H; and

R³³, R³⁶, R⁴¹ and R⁴⁴ represent H.

### Example 41

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₃—O—C(═O)—NR³¹R³²;

R⁹ represents —C₂H₅;

R¹⁰ represents —C₂H₅;

R³¹ represents —CH₂—(CH₂OCH₂)₄₋₅CH₂—O—CH₃;

R³² represents H; and

R³³, R³⁶, R⁴¹ and R⁴⁴ represent H.

### Example 42

R¹ represents —(CH₂)₂—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents —CH₂—(CH₂OCH₂)₄₋₅CH₂—O—CH₃;

R³² represents H; and

R³³, R³⁶, R⁴¹ and R⁴⁴ represent H.

### Example 43

R¹ represents —(CH₂)₁—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₂—O—C(═O)—NR³¹R³²;

R⁹ represents —C₂H₅;

R¹⁰ represents —C₂H₅;

R³¹ represents —CH₂—(CH₂OCH₂)₄₋₅CH₂—O—CH₃;

R³² represents H; and

R³³, R³⁶, R⁴¹ and R⁴⁴ represent H.

### Example 44

R¹ represents —(CH₂)₁—O—C(═O)—NR³¹R³²;

R² represents —CH₃;

R³ represents —C₂H₅;

R⁴ represents —C₂H₅;

R⁵ represents —C₂H₅;

R⁶ represents —C₂H₅;

R⁷ represents —CH₃;

R⁸ represents —(CH₂)₁—O—C(═O)—NR³¹R³²;

R⁹ represents —C₂H₅;

R¹⁰ represents —C₂H₅;

R³¹ represents —CH₂—(CH₂OCH₂)₄₋₅CH₂—O—CH₃;

R³² represents H; and

R³³, R³⁶, R⁴¹ and R⁴⁴ represent H.

### Materials and Methods

Cell Lines, Growth Conditions and Animal Xenograft Model

All cell lines were grown in RPMI 1640 with 10% fetal bovine serum. Cells were treated at a density of 100,000 cells/ml with sapphyrins for 24 hrs and then assessed for apoptosis. Some cells were cultured for up to 96 hrs and then assessed for growth inhibition by counting cells using a coulter counter. For the xenograft model, 10 million Ramos cells were injected subcutaneously into the right hind flank of CD-1 nude mice that had been irradiated with 3 Gy 24 hrs prior to tumor implantation. Seven days later, the mice were treated with sapphyrin given intravenously in the tail vein q day×2 doses. Some animals were sacrificed the next day for analysis of drug uptake in tumor and spleen, tumor cell killing and tumor cell culture.

Apoptosis Assays

Annexin binding and propidium iodide exclusion were assayed using reagents from Biosource (Camarillo, Calif.) per manufacturer's protocol.

Caspase-3 activity was assayed using the EnzChek Caspase-3 Assay Kit #2 (Molecular Probes, Eugene, Oreg.). Cells were harvested, rinsed in cold PBS, and lysed, and supernatants were quantitated. Cell lysates were analyzed according to the manufacturer's protocol. Reactions were incubated in a reaction mixture containing Z-DEVD-R110 (0.5 mM) at room temperature for 30 minutes, and fluorescence levels were determined at an excitation of 485 nm and emission of 510 nm using a fluorescence plate reader. For each cell line, measured fluorescence levels were normalized to fluorescence levels of non-treated cell lysates.

Western Blotting

Cells were lysed in triple-detergent lysis buffer [50 mM Tris-Cl (pH 8.0), 150 mM NaCl, 0.1% SDS, 0.5% deoxycholic acid, 1.0% NP-40, supplemented with 100 mM PMSF and protease inhibitor cocktail] on ice for 10 minutes. After centrifugation at 10,000×g for 10 min, supernatants were quantified for protein amount and equal quantities of protein were resolved on the appropriate percentage SDS-polyacrylamide gels (Bio-Rad, Hercules, Calif.). Gels were transferred to polyvinylidene difluoride membrane using a Bio-Rad Semi-dry Transfer Cell (Bio-Rad, Hercules, Calif.). Western blotting was performed using primary and alkaline phosphatase-conjugated secondary antibodies specified in the text. Antibodies to caspases and PARP specifically recognized the full-length and cleaved forms of their respective antigens (Cell Signaling Technologies, Beverly, Mass.). Protein bands were detected using ECF fluorescent substrate (Amersham Biosciences, Piscataway, N.J.). All membranes were blotted with an anti-tubulin antibody (Sigma) to control for loading and transfer. Bands were imaged and quantitated in the linear range and normalized to tubulin using the Typhoon 8600 Variable Mode Imager (Amersham Biosciences, Piscataway, N.J.).

### Results

Cytotoxicity of Formula I Compounds

| | % Annexin positive cells after | | | | After | Animal |
|---|---|---|---|---|---|---|
| | 24 hrs (sapphyrin dose) | | | | 96 hr | toxicology |
| Compound | 0.5 Um | 1 uM | 2.5 uM | 5 uM | 0.5 uM | uM/kg³ |
| Ex. 1 | Back | 38 | 99 | sat | 91 | 1/9 dead at |
| | | | | | | 26.8 (22.3) |
| Ex. 1 | Back | 30 | 96 | NT | 52 | As above |
| Ex.2 | Back | 10 | 41 | 97 | 13 | 4/6 dead |
| | | | | | | at 40 (30) |
| Ex. 3 | 10.6 | 93 | sat | sat | | |
| Ex. 4 | Back | Back | back | back | | |
| Ex. 5 | 92 | 99.9 | sat | sat | NT-all | LD-30 uM/kg |
| | | | | | cells | 10 uM/kg |
| | | | | | dead | |
| Phthalimide | back | 16 | | sat | GI-50% | |

1 Back = background
2 Sat = saturated assay conditions: killing greater 95%
3 Numbers in parentheses are highest doses that showed no deaths from GB
4 GI = growth inhibition due to sapphyrin compound 96 hrs post treatment compared to control untreated cells

The following data is for the following compound of Formula I, Example 11:

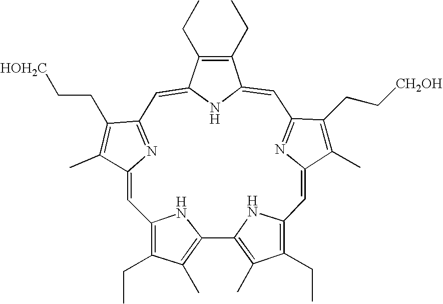

### Example 11

### Inhibition of A549 Human Lung Cancer Cell Survival by Sapphyrin

The clonogenic survival of A549 human lung cells was used to assess the activity of sapphyrins under cell culture conditions. A549 cells (7.5×10⁴ cells per flask) in RPMI medium supplemented with 15% fetal bovine serum were allowed to adhere overnight to T-25 flasks. Stock sapphyrin, as a 5 mM solution in DMSO, was added to give the final sapphyrin concentrations indicated in FIG. 1. The cultures were incubated at 37° C. under a 5% CO2/95% air atmosphere for 24 hours. Cultures were washed once with Hank's balanced salt solution (HBSS), and 0.05% w/v trypsin, 0.5 mM EDTA solution in HBSS was added to form a cell suspension. Trypsin was inhibited by addition of RPMI medium supplemented with 15% fetal bovine serum, the cell suspension was transferred to a centrifuge tube, and the tube was centrifuged for 5 minutes at 500×g. The resulting cell pellet was resuspended in fresh medium and counted using a Coulter counter. Cells were sub-cultured in T-25 flasks in 7 mL RPMI medium supplemented with 15% fetal bovine serum. Flasks were incubated at 37° C. under a 5% CO₂/95% air atmosphere for 12 days, whereupon medium was removed, and colonies of cells were fixed by addition of 2-propanol (7 mL) for 20 minutes. The 2-propanol was removed, the flasks were rinsed thrice with water, and colonies were stained with 1% aqueous crystal violet solution for 20 minutes. Crystal violet was removed, flasks were rinsed thrice with water (3×7 mL), and then allowed to air dry. Colonies were counted using a low power microscope. A dose-response was observed towards Example 11.

[image]

Cytoxicity of Example 12

Cytotoxicity was evaluated using Annexin-V staining and caspase activation as markers of apoptosis. Cell lines were grown in RPMI 1640 with 10% heat inactivated fetal bovine serum. Example 12 was added to cell cultures in concentrations ranging from 100 nM-5 μM.

Caspase-3 activity was assayed using the EnzChek Caspase-3 Assay Kit #2 (Molecular Probes, Eugene, Oreg.). Cell lysates were analyzed according to the manufacturer's protocol. For each cell line, measured fluorescence levels were normalized to fluorescence levels of non-treated cell lysates.

Cells were lysed and supernatants were quantified for protein amount and equal quantities of protein were resolved on the appropriate percentage SDS-polyacrylamide gels (Bio-Rad, Hercules, Calif.). Gels were transferred to polyvinylidene difluoride membrane, and western blotting was performed using primary and alkaline phosphatase-conjugated secondary antibodies specified in the figures. Antibodies to caspases and PARP specifically recognized the full-length and cleaved forms of their respective antigens (Cell Signaling Technologies, Beverly, Mass.). Protein bands were detected using ECF fluorescent substrate (Amersham Biosciences, Piscataway, N.J.). All membranes were blotted with an anti-tubulin (Sigma) or anti-actin (Santa Cruz Biotechnology, Inc.) antibody to control for loading and transfer. Bands were imaged and quantified in the linear range and normalized to tubulin, or actin, using the Typhoon 8600 Variable Mode Imager (Amersham Biosciences, Piscataway, N.J.).

Cytoxocity for Example 5

Tumor xenograft studies were performed in irradiated CD-1 Nude mice using Ramos lymphoma cells (1×10⁷ cells) injected into the hind flank. Sapphyrin injections into the tail vein were performed on days 9 and 10 and tumors were harvested on day 11 of the protocol. Drug uptake (Becton Dickinson FACSCalibur), Annexin-V staining and caspase-3 activity assays were performed on fresh tumor. Tumor cells were then cultured and assessed for Annexin-V binding and counted to monitor growth. Drug uptake was also monitored by near infrared fluorescence using a LI-COR Odyssey scanner. Compound of Example 5 was also evaluated for tumor growth delay in both a minimal disease model (2 doses of Example 5, 3 days after tumor implantation) and an established tumor model (2 doses of Example 5, 7 and 8 days after tumor implantation, when tumors were palpable). Tumor sizes were measured at least every other day. Tumor volume was calculated assuming the conformation of a hemi ellipsoid: V=π/6×length×width×height.

Definitions

As used here in, the following terms are intended to have the respective meaning as defined.

Alkyl: The term “alkyl” as used herein in intended to include a straight chain alkyl group having up to four carbon atoms and a brancked alkyl group having up to six carbon atoms. Illustrative examples of such groups are methyl, ethyl, butyl, isopropyl, isobutyl, isopentyl and the like.

Pharmaceutically Acceptable Salt: The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and triamines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically acceptable acid addition salts may be prepared from inorganic and organic acids. The inorganic acids that can be used include hydrochloric acid, hydrobromic acid, sulfuric acid, nitric acid, phosphoric acid, and the like. The organic acids that can be used include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Examples of such pharmaceutically acceptable salts are the iodide, acetate, phenyl acetate, trifluoroacetate, acryl ate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, g-hydroxybutyrate, b-hydroxybutyrate, butyne-1,4-dioate, hexyne-'1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like of a compound of formula I.

By “pharmaceutically acceptable” it is also meant that in a formulation containing the compound of formula I, the carrier, diluent, excipients, and salt must be compatible with the other ingredients of the formulation, and not deleterious to the recipient thereof.

Prodrugs: “Prodrugs” are derivatives of the compounds of the invention which have metabolically cleavable groups and become by solvolysis or under physiological conditions the compounds of the invention which are pharmaceutically active in vivo. For example, ester derivatives of compounds of this invention are often active in vivo, but not in vitro. Other derivatives of the compounds of this invention have activity in both their acid and acid derivative forms, but the acid derivative form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with an amine. Simple aliphatic or aromatic esters derived from acidic groups pendant on the compounds of this invention are preferred prodrugs. In some cases it is desirable to prepare double ester type prodrugs such as (acyloxy)alkyl esters or ((alkoxycarbonyl)oxy)alkyl esters.

Therapeutically Effective Amount: The term “therapeutically effective amount”, as used herein refers to an amount of drug that is safe and produces the necessary therapeutic effect. This amount can be determined by safety studies in animals and human hosts, and efficacy studies in animal and human hosts. Procedures for such studies are well known to one skilled in the art.

Halogen: The term “halogen” represents Cl, Br, I and/or F.

1. A compound of Formula I 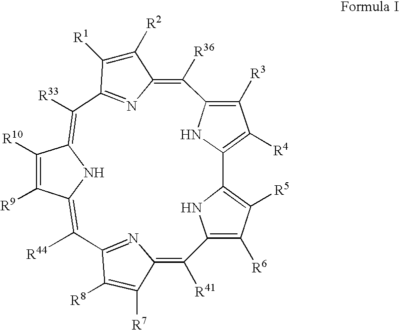
its pharmaceutically acceptable salts and prodrugs there of, wherein:
R¹ represents —(CH₂)₁₋₄—O—C(═O)—NR³¹R³², —(CH₂)₁₋₄—X—CH₂—O—(CH₂CH₂O)₀₋₃—CH₃, —C₁₋₄ alkyl, —(CH₂)₁₋₄—R²¹, H. —R²¹, or —(CH₂)₁₋₄—OH;
R² represents H, —C₁₋₄ straight chain alkyl, or —C₃₋₆ branched alkyl;
R³ represents H, —C₁₋₄ straight chain alkyl, —C₃₋₆ branched alkyl, halogen, —NO₂, —CN, O-alkyl, —(CH₂)₁₋₄O—(CH₂)₁₋₄O—(CH₂)₁₋₄O—(CH₂)₀₋₂—CH₃, —(CH₂)₁₋₄—OH, or —(CH₂)₁₋₄—OCOCH₃;
R⁴ represents H, —C₁₋₄ straight chain alkyl, —C₃₋₆ branched alkyl, halogen, —NO₂, —CN, —O-alkyl, —(CH₂)₁₋₄—OH, —(CH₂)₁₋₄O—(CH₂)₁₋₄O—(CH₂)₁₋₄O—(CH₂)₀₋₂—CH₃, or —(CH₂)₁₋₄—OCOCH₃;
R⁵ represents H, —C₁₋₄ straight chain alkyl, —C₃₋₆ branched alkyl, halogen, —NO₂, —CN, —O-alkyl, —(CH₂)₁₋₄—OH, —(CH₂)₁₋₄O—(CH₂)₁₋₄O—(CH₂)₁₋₄O—(CH₂)₀₋₂—CH₃, or —(CH₂)₁₋₄—OCOCH₃;
R⁶ represents H, C₁₋₄ straight chain ally, C₃₋₆ branched alkyl, halogen, NO₂, —CN, O-alkyl, —(CH₂)₁₋₄—OH, —(CH₂)₁₋₄O—(CH₂)₁₋₄O—(CH₂)₁₋₄O—(CH₂)₀₋₂—CH₃, or —(CH₂)₁₋₄—OCOCH₃;
R⁷ represents H, —C₁₋₄ straight chain alkyl, or —C₃₋₆ branched alkyl;
R⁸ represents —(CH₂)₁₋₄—X—CH₂—O—(CH₂CH₂O)₀₋₃—CH₃, —C₁₋₄ alkyl, —(CH₂)₁₋₄—R²¹, —R²¹, H, —(CH₂)₁₋₄—O—C(═O)—NR³¹R³², or (CH₂)₁₋₄—OH;
R⁹ represents —C₁₋₄ straight chain alkyl, —C₃₋₆ branched alkyl, H, —O—C₁₋₄-alkyl, —O—C₃₋₆ branched alkyl, or —(CH₂)₁₋₄O—(CH₂)₁₋₄O—(CH₂)₁₋₄O—(CH₂)₀₋₂—CH₃;
R¹⁰ represents H, —C₁₋₄ straight chain alkyl, —C₃₋₆ branched alkyl; —O—C₁₋₄-alkyl, or —O—C₃₋₆ branched alkyl; X represents —OCO₂CH₂—, —O₂C—, —NHCO—, —OCONHCH₂, —NHCO₂CH₂—, —NHCONHCH₂—, or —NHCH₂—;
R²¹, R²², R²³, R²⁴, and R²⁵ independently at each occurrence are selected from H, —CH₂OH, —CH₂NH₂, —CH₂N(C₂H₄OH)₂, —COOH, —CON(C₂H₄OH)₂, —OCON(C₂H₄OH)₂, —NHCON(C₂H₄OH)₂, and —O(CH₂CH₂O)₀₋₃CH₃;
R³¹ represents H, —(CH₂)₁₋₆OH, C((CH₂)₁₋₄OH)₃, —C((CH₂)₁₋₄O-alkyl)₃, —(CH₂)₁₋₆O-alkyl, or —(CH₂)₁₋₄O—(CH₂)₁₋₄O—(CH₂)₁₋₄O—(CH₂)₀₋₂—CH₃;
R³² represents H, —(CH₂)₁₋₆OH, —C((CH₂)₁₋₄OH)₃, —C((CH₂)₁₋₄O-alkyl)₃, —(CH₂)₁₋₆O-alkyl, or —(CH₂)₁₋₄O—(CH₂)₁₋₄O—(CH₂)₁₋₄O—(CH₂)₀₋₂—CH₃;
R³³ represents H, —C₁₋₄ alkyl, —O—C₁₋₄-alkyl, —O—C₃₋₆ branched alkyl, or 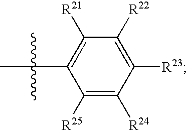
R³⁶ represents H or —C₁₋₄ alkyl;
R³⁷ represents H or —C₁₋₄ alkyl;
R⁴¹ represents H or —C₁₋₄ alkyl; and
R⁴⁴ represents H, —C₁₋₄ alkyl, —O—C₁₋₄ alkyl, or

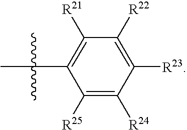

2. A compound of claim 1 wherein:
R¹ represents —(CH₂)₃—O—C(═O)—NR³¹R³²;
R² represents —C₁₋₄ straight chain alkyl, or —C₃₋₆ branched alkyl;
R³ represents —C₁₋₄ straight chain alkyl, —C₃₋₆ branched alkyl, halogen, —(CH₂)₁₋₄O—(CH₂)₁₋₄O—(CH₂)₁₋₄O—(CH₂)₀₋₂—CH₃, —O-alkyl, (CH₂)₁₋₄—OH, or —(CH₂)₁₋₄—OCOCH₃;
R⁴ represents —C₁₋₄ straight chain alkyl, —C₃₋₅ branched alkyl, halogen, —(CH₂)₁₋₄—OH, or —(CH₂)₁₋₃—OCOCH₃;
R⁵ represents —C₁₋₃ straight chain alkyl, —C₃₋₅ branched alkyl, halogen, —O-alkyl, —(CH₂)₁₋₃—OH, —(CH₂)₁₋₄O—(CH₂)₁₋₄O—(CH₂)₁₋₄O—(CH₂)₀₋₂—CH₃, or —(CH₂)₁₋₃—OCOCH₃;
R⁶ represents —C₁₋₃ straight chain alkyl, —C₃₋₅ branched alkyl, halogen, —O-alkyl, —(CH₂)₁₋₃—OH, —(CH₂)₁₋₃O—(CH₂)₁₋₄O—(CH₂)₁₋₄O—(CH₂)_{0.2}—CH₃, or —(CH₂)₁₋₄—OCOCH₃;
R⁷ represents —C₁₋₃ straight chain alkyl, or —C₃₋₅ branched alkyl;
R⁸ represents —(CH₂)₂₋₄—O—C(═O)—NR³¹R³²;
R⁹ represents —C₁₋₃ straight chain alkyl, —C₃₋₅ branched alkyl, —(CH₂)₂₋₄O—(CH₂)₁₋₄O—(CH₂)₁₋₄O—(CH₂)₀₋₂—CH₃, or —O-alkyl;
R¹⁰ represents —C₁₋₄ straight chain alkyl, —C₃₋₆ branched alkyl, or —O-alkyl;
R³¹ represents H, or —(CH₂)₂₋₄O—(CH₂)₁₋₄O—(CH₂)₁₋₄O—(CH₂)₀₋₂—CH₃; and
R³² represents H, or —(CH₂)₂₋₄O—(CH₂)₁₋₄O—(CH₂)₁₋₄O—(CH₂)₀₋₂-CH₃.

3. A compound of claim 1 wherein:
R² represents —CH₃;
R³ represents —CH₃, —C₂H₅, or —OCH₃;
R⁴ represents —CH₃, or —C₂H₅;
R⁵ represents —CH₃, —C₂H₅, or —OCH₃;
R⁶ represents —CH₃, —C₂H₅, or —OCH₃;
R⁷ represents —CH₃;
R⁹ represents —CH₃, —C₂H₅, or —OCH₃;
R¹⁰ represents —CH₃, —C₂H₅, or —OCH₃;
R³¹ represents —(CH₂)₂—O—(CH₂)₂—O—(CH₂)₂—O—CH₃;
R³² represents —(CH₂)₂—O—(CH₂)₂—O—(CH₂)₂—O—CH₃; and
R³³, R³⁶, R⁴¹ and R⁴⁴ represent H.

4. A compound of claim 1, wherein:
R¹ represents —(CH₂)₃—O—C(═O)—NR³¹R³²;
R² represents —CH₃;
R³ represents —CH₃, or —C₂H₅;
R⁴ represents —CH₃, or —C₂H₅;
R⁵ represents —CH₃, or —C₂H₅;
R⁶ represents —CH₃, —C₂H₅, or —OCH₃;
R⁷ represents —CH₃;
R⁹ represents —CH₃, —C₂H₅, or —OCH₃;
R¹⁰ represents —CH₃, —C₂H₅, or —OCH₃;
R³¹ represents —(CH₂)₂—O—(CH₂)₂—O—(CH₂)₂—O—CH₃;
R³² represents —(CH₂)₂—O—(CH₂)₂—O—(CH₂)₂—O—CH₃; and
R³³, R³⁶, R⁴¹ and R⁴⁴ represent H.

5. A compound of claim 1 wherein:
R¹ represents —(CH₂)₁₋₃—O—C(═O)—NR³¹R³²;
R² represents —CH₃;
R³ represents —C₂H₅;
R⁴ represents —CH₃;
R⁵ represents —CH₃;
R⁶ represents —C₂H₅;
R⁷ represents —CH₃;
R⁸ represents —(CH₂)₁₋₃—O—C(═O)—NR³¹R³²;
R⁹ represents —C₂H₅;
R¹⁰ represents —C₂H₅;
R³¹ represents —(CH₂—CH₂O)₃CH₃;
R³² represents —(CH₂—CH₂O)₃CH₃; and
R³³, R³⁶, R⁴¹ and R⁴⁴ represent H.

6. A compound of claim 1, wherein:
R¹ represents —(CH₂)₁₋₃—O—C(═O)—NR³¹R³²;
R² represents —CH₃;
R³ represents —C₂H₅;
R⁴ represents —C₂H₅;
R⁵ represents —C₂H₅;
R⁶ represents —C₂H₅;
R⁷ represents —CH₃;
R⁸ represents —(CH₂)₁₋₃—O—C(═O)—NR³¹R³²;
R⁹ represents —C₂H₅;
R¹⁰ represents —C₂H₅;
R³¹ represents —(CH₂—CH₂O)₃CH₃;
R³² represents —(CH₂—CH₂O)₃CH₃; and
R³³, R³⁶, R⁴¹ and R⁴⁴ represent H.

7. A compound of claim 1, wherein:
R¹ represents —(CH₂)₂—O—C(═O)—NR³¹R³²;
R² represents —CH₃;
R³ represents —C₂H₅;
R⁴ represents —C₂H₅;
R⁵ represents —C₂H₅;
R⁶ represents —C₂H₅;
R⁷ represents —CH₃;
R⁸ represents —(CH₂)₂—O—C(═O)—NR³¹R³²;
R⁹ represents —C₂H₅;
R¹⁰ represents —C₂H₅;
R³¹ represents —(CH₂)₂OH;
R³² represents —(CH₂)₂OH; and
R³³, R³⁶, R⁴¹ and R⁴⁴ represent H.

8. A compound of Formula I: 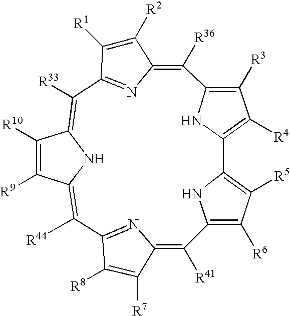
wherein:
R¹ represents —(CH₂)₂—O—C(═O)—NR³¹R³²;
R² represents —CH₃;
R³ represents —CH₃, —C₂H₅, or —OCH₃;
R⁴ represents —CH₃, or —C₂H₅;
R⁵ represents —CH₃, —C₂H₅, or —OCH₃;
R⁶ represents —CH₃, —C₂H₅, or —OCH₃;
R⁷ represents —CH₃;
R⁸ represents —(CH₂)₂—O—C(═O)—NR³¹R³²;
R⁹ represents —CH₃, —C₂H₅, or —OCH₃;
R¹⁰ represents —CH₃, —C₂H₅, or —OCH₃;
R³¹ represents —(CH₂)₂—O—(CH₂)₂—O—(CH₂)₂—O—CH₃;
R³² represents —(CH₂)₂—C—(CH₂)₂—O—(CH₂)₂—O—CH₃; and
R³³, R³⁶, R⁴¹ and R⁴⁴ represent H.

9. A compound of Formula I: 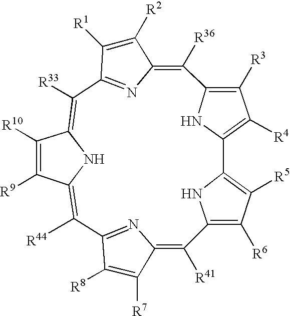
wherein:
R¹ represents —(CH₂)₂O—C(═O)—NR³¹R³²;
R² represents —CH₃;
R³ represents —C₂H₅, or —OCH₃;
R⁴ represents —CH₃;
R⁵ represents —CH₃;
R⁶ represents —C₂H₅, or —OCH₃;
R⁷ represents —CH₃;
R⁸ represents —(CH₂)₂—O—C(═O)—NR³¹R³²;
R⁹ represents —CH₃, —C₂H₅, or —OCH₃;
R¹⁰ represents —CH₃, —C₂H₅, or —OCH₃;
R³¹ represents —(CH₂)₂—O—(CH₂)₂—O—(CH₂)₂—O—CH₃;
R³² represents —(CH₂)₂—O—(CH₂)₂—O—(CH₂)₂—O—CH₃; and
R³³, R³⁶, R⁴¹ and R⁴⁴ represent H.

10. A compound of Formula I: 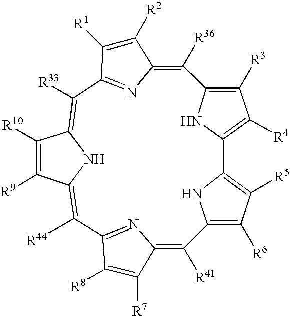
wherein:
R¹ represents —(CH₂)₂—O—C(═O)—NR³¹R³²;
R² represents —CH₃;
R³ represents —C₂H₅;
R⁴ represents —CH₃;
R⁵ represents —CH₃;
R⁶ represents —C₂H₅;
R⁷ represents —CH₃;
R⁸ represents —(CH₂)₂—O—C(═O)—NR³¹R³²;
R⁹ represents —C₂H₅;
R¹⁰ represents —C₂H₅;
R³¹ represents —(CH₂)₂—O—(CH₂)₂—O—(CH₂)₂—O—CH₃;
R³² represents —(CH₂)₂—O—(CH₂)₂—O—(CH₂)₂—O—CH₃; and
R³³, R³⁶, R⁴¹ and R⁴⁴ represent H.

11. A pharmaceutical composition, comprising a pharmaceutically acceptable carrier and a therapeutically effective amount of a compound of claim 1 or a pharmaceutically acceptable salt form thereof.

12. A pharmaceutical composition, comprising a pharmaceutically acceptable carrier and a therapeutically effective amount of a compound of claim 8 or a pharmaceutically acceptable salt form thereof.

13. A pharmaceutical composition, comprising a pharmaceutically acceptable carrier and a therapeutically effective amount of a compound of claim 9 or a pharmaceutically acceptable salt form thereof.

14. A pharmaceutical composition, comprising a pharmaceutically acceptable carrier and a therapeutically effective amount of a compound of claim 10 or a pharmaceutically acceptable salt form thereof.

15. A pharmaceutical composition, comprising a pharmaceutically acceptable carrier and a therapeutically effective amount of a compound of claim 5 or a pharmaceutically acceptable salt form thereof.

16. A pharmaceutical composition, comprising a pharmaceutically acceptable carrier and a therapeutically effective amount of a compound of claim 6 or a pharmaceutically acceptable salt form thereof.

17. A method of treating a host harboring a neoplasm comprising administering to the host a Formula I compound of claim 1.

18. A method of treating a host harboring a neoplasm comprising administering to the host a Formula I compound of claim 5.

19. A method of treating a host harboring a neoplasm comprising administering to the host a Formula I compound of claim 6.

20. A method of treating a host harboring a neoplasm comprising administering to the host a Formula I compound of claim 8.

21. A method of treating a host harboring a neoplasm comprising administering to the host a Formula I compound of claim 9.

22. A method of treating a host harboring a neoplasm comprising administering to the host a Formula I compound of claim 10.
